(19) United States
(12) Patent Application Publication
(10) Pub. No.: US 2004/0034311 A1
(43) Pub. Date: Feb. 19, 2004

(54) Guidewire with viewing capability

(76) Inventor: Albert Mihalcik, Hollis, NH (US)

Correspondence Address: KIRKPATRICK & LOCKHART LLP, 75 STATE STREET, BOSTON, MA, 02109-1808, US

(21) Appl. No.: 10/275,425

(22) PCT Filed: May 18, 2001
(86) PCT No.: PCT/US01/16063

(51) Int. Cl. A61B 5/00

(52) U.S. Cl. 600/585

(57) ABSTRACT

A guidewire having a shaft comprising an optical fiber and an optical handle through which direct visualization is provided. The guidewire may be inserted alone through a passageway of a patient to visualize the internal anatomy of the patient such as inside a passageway or the inside of an organ. The guidewire may also be inserted through a catheter or endoscope to aid in navigation to the intended location. Additionally, the guidewire may be inserted into other catheters, which are then tracked over the guidewire to the subject area The guidewire comprises a removable optical handle which permits catheter exchanges over its proximal end when made in an exchange length configuration or is used with rapid-exchange catheters. The guidewire may also comprise a light fiber bundle along with the optical fiber, together surrounded by a jacket to form the elongate guidewire.

### FIELD OF THE INVENTION

The present invention relates to guidewires and to medical devices providing direct viewing capability to the internal anatomy of the human body.

### BACKGROUND OF THE INVENTION

A challenge in the exploration and treatment of internal areas of the human anatomy has been adequately visualizing the area of concern. Visualization can be especially troublesome in minimally invasive procedures in which small diameter, elongate instruments, such as catheters and endoscopes are navigated through natural passageways of a patient to an area of concern either in the passageway or in an organ reachable through the passageway. The injection of contrast media may also serve to irritate the organ of concern, thus necessarily limiting the amount of contrast that safely can be injected into the area.

Radiography has provided some assistance to practitioners by providing radiological images of internal anatomy during various medical procedures. In such procedures a radiographical image of the area of concern is obtained from outside the patient while medical instruments having radiopaque markers are navigated through passageways of the patient and radiopaque contrast media is injected into the passageway to visualize the anatomy. Disadvantages of such procedures are exposure to X-ray radiation, both to the patient and medical personnel and insufficient visual detail of the targeted area. Because the radiographical image is black and white, features of the anatomy and the medical instruments appear only as dark or light images. Additionally, because the image is obtained from outside the body, close inspection of the anatomy is not possible and depth perception and orientation can make navigation and analysis of the image challenging.

More detailed information regarding the anatomy can be discerned from direct viewing of the anatomy provided through one or more of the elongate instruments used in the procedure. Various types of endoscopes configured for use in various passageways of the body such as the esophagus, rectum or bronchus can be equipped with direct viewing capability through the use of optical fibers extending through the length of the scope. However, because endoscopes also provide a working channel through which other medical instruments must pass, lighting bundles and components to provide steering capability at its distal end, the scope is typically of a relatively large diameter. The large diameter limits the use of the endoscope to relatively large body lumens and prohibits their use in smaller ducts and organs that branch from a large body lumen. Typically, the endoscope is used to get close to a smaller passageway or region of concern and another instrument, such as a catheter, is then extended through the working channel of the endoscope and into the smaller passageway. Although the endoscope provides direct visualization of the large body passageway and entrance to adjoining ducts and lumens, after the smaller catheter has been extended from the endoscope into the smaller duct or lumen, direct visualization is unavailable and the physician must again rely on radiographical means to visualize the area of concern or probe blindly.

It would be desirable to provide a small diameter device that provides direct viewing capability of internal anatomy such as ducts, vessels and organs of the human body that is easy to use with other minimally invasive medical instruments.

### SUMMARY OF THE INVENTION

The present invention provides a guidewire having viewing capability and a relatively small diameter that may be inserted into areas of internal anatomy of a patient to provide direct visualization of those areas for diagnosis and treatment of various medical conditions. Anatomical structures into which the present viewing guidewire may be introduced include any type of duct, lumen, vessel, orifice, internal cavity or organ. The guidewire is comprised of an optical fiber, a light transmitting fiber bundle and a jacket encircling the fibers to form a shaft having proximal and distal ends. Additionally, the guidewire comprises an optical handle that can be made removable to permit catheter exchanges over the proximal end of the guidewire while maintaining the guidewire in position in a passageway.

The optical handle comprises a series of lenses which project the image transmitted through the optical fiber to an image output means such as an eyepiece at the proximal end of the optical handle. Images may be viewed directly through the eyepiece or the eyepiece may be mounted in a coupling that fixes the optical handle to a lens of a video camera so that the images may be manipulated by the zoom and focus features of the camera and projected onto a video monitor screen. The optical handle may be disassembled into several separate components including a releasable securement mechanism, such as a collet, which releases the handle from the guidewire shaft and a fiber optic light source connector which joins the light transmitting fiber bundle to a fiber optic light source. A variety of alternative securement mechanisms are also possible.

The guidewire may be made to have a relatively small diameter, typical of commercially available guidewires, such as 0.035 inch. The guidewire may be made even smaller, such as 0.025 or 0.018 inch, by reducing the size of the optical fiber element. The small size of the guidewire permits it to be inserted through a wide variety of body passageways and cavities, as well as through lumens of commercially available catheters and endoscopes. The guidewire of the present invention may be used as any other commercially available guidewire would be used but provides the additional feature of direct viewing capability from its distal tip. The flexibility of the guidewire may be modified to provide for the anatomy of various areas of the body by changing the jacket material or adding a combination of materials to form a composite jacket for the guidewire.

The light transmitting fiber bundle extends parallel to the optical fiber, in a side-by-side relationship, within the jacket through most of the length of the guidewire. Preferably, near the distal end of the guidewire, the fibers of the light bundle are spread apart and moved to surround the optical fiber element. The resulting distal face of the guidewire comprises the optical fiber in the center, surrounded by a ring of light fiber bundle elements. This arrangement provides full illumination around the circumference of the optical fiber.

In use with an endoscope having viewing capability, the guidewire of the present invention provides medical personnel with two image views of the procedure. Two images can be especially useful in procedures such as biliary procedures. In a biliary procedure, an endoscope having viewing capability is navigated through the esophagus and the duodenum to be adjacent to the papilla at the exit of the common bile duct. The guidewire having viewing capability may be advanced through the working channel of the endoscope, alone or in combination with a catheter to be navigated into the papilla. The viewing capability of the guidewire permits the physician to see the papilla during attempts to cannulate it with the guidewire. The added visual capability may reduce the difficulty in cannulating the papilla, reducing the incidence of traumatizing the papilla, which can otherwise delay the procedure and further complicate cannulation. Once the guidewire and catheter have entered the papilla and are advanced into the biliary tree, the image provided from the endoscope can be used to monitor the position of the catheter and guidewire, visually, while the guidewire provides images from deep in the common bile duct and from inside the other ducts and organs which branch from the common bile duct.

The guidewire of the present invention may also be used in combination with a mother/daughter type endoscope system to provide three concurrent images of a medical procedure. In that situation, the guidewire would be placed through the daughter scope and extended beyond to provide images from the most distal regions of the anatomy, which are typically of smaller diameter than either the daughter or mother scope. Each of the daughter and mother scopes may then be used to provide images along the procedure path that are more proximal to the position of the guidewire. The guidewire provides advantages over a mother and daughter scope system alone in that its diameter is smaller than that of a daughter scope permitting it to reach and enter smaller passageways and organs of a patient. Additionally, the guidewire of the present invention permits other catheters and elongate instruments to be exchanged over it.

The guidewire having viewing capability makes possible more accurate diagnosis and treatment of a wide range of internal areas of the body in addition to the biliary system. For example, the guidewire of the present invention may be inserted into the fallopian tubes of a patient to visually inspect and detect early stages of cancer. The wire may be used in the urinary system, through a mother/daughter scope arrangement to examine for cysts or other abnormalities. In the chest, the guidewire with viewing capability can be used to enter with accuracy the subsegments of the bronchus, heretofore entered blindly with currently available instruments. Once placed in the bronchus, the guidewire serves as a track over which cytology brushes or needles may be guided to accurately obtain tissue samples from areas suspected of having tumors.

The guidewire of the present invention is also useful for verifying accurate stent placement in various passageways of the human body. In a stent delivery procedure, the guidewire may be navigated to a treatment site within a passageway. A balloon catheter carrying a balloon expandable stent may then be delivered over the guidewire to the treatment site. Positioning of the balloon catheter and stent may be verified by the direct visualization provided by the guidewire. After the stent is positioned, it may be expanded by the balloon catheter and the wire and catheter withdrawn slightly, the wire then can be used to visualize the expanded stent from inside of the stent to verify proper positioning of the stent within the passageway.

Another feature of the guidewire of the present invention is utility as a biliary manometry device. The optical fiber used to provide a viewing capability may also be used to transmit pressure readings from the distal end of the guidewire to its proximal end. For such use, the guidewire is equipped with one or more pressure sensors at its distal end in communication with individual elements of the optical fiber. At the proximal end of the wire, the individual elements of the optical fiber are joined to pressure transducers so that pressure can be sensed and measured and displayed. In use, the guidewire is inserted into the common bile duct and navigated to a position that places the several pressure sensors in zones along the length of the common bile duct where pressure is to be measured. Because the optical fiber of the guidewire contains many separate elements, numerous pressure sensors and transducers can be used, such as six to ten or more. The increase in pressure monitoring sites, increased from three, typically found in a manometry catheter, reduces time and provides more accurate information about the common bile duct to the physician.

It is an object of the present invention to provide a guidewire having viewing capability that is sized to be used in the passageways and organs of a patient and with the devices that a conventional guidewire would be used.

It is another object of the invention to provide a guidewire having viewing capability and a removable optical handle that permits catheters to be exchanged over the wire.

It is another object of the invention to provide a guidewire that may function to perform biliary manometry.

It is another object of the invention to provide a method of viewing a passageway of a patient using a guidewire having viewing capability.

It is another object of the present invention to provide a method of examining an internal organ using a guidewire having viewing capability.

It is another object of the present invention to provide a method of cannulating the papilla using a guidewire having a viewing capability.

It is another object of the invention to provide a method of positioning a stent in a passageway of the body using a guidewire having viewing capability.

### BRIEF DESCRIPTION OF THE DRAWINGS

The foregoing and other objects and advantages of the invention will be appreciated more fully from the following further description thereof, with reference to the accompanying diagrammatic drawings wherein:

FIG. 1 is a diagrammatic assembly illustration of the guidewire of the present invention with detachable optical handle;

FIG. 1A shows an end view of the guidewire of the present invention; and

FIG. 2 is a diagrammatic illustration of a guidewire of the present invention being introduced into the biliary system;

FIGS. 3-9 are diagrammatic sectional illustrations of alternate optical handle releasable securement mechanisms.

### DESCRIPTION OF THE ILLUSTRATIVE EMBODIMENTS

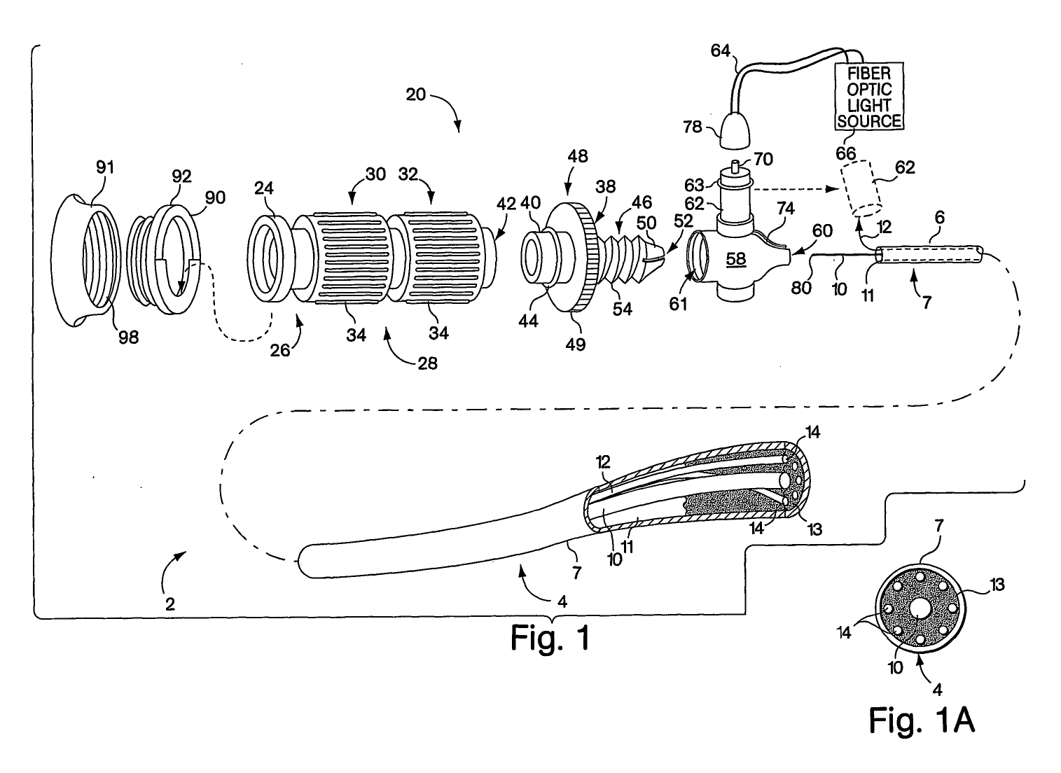

FIG. 1 shows a diagrammatic assembly drawing 2 of a viewing guidewire of the present invention. The guidewire 2 comprises a shaft 4 having a proximal end 6 and distal end 8, which comprises a tubular jacket 7. Contained within a lumen 11 of the tubular shaft 4, in parallel relation over most of the shaft length, are an optical fiber 10 for providing viewing capability to the guidewire and a light transmitting fiber bundle 12 for transmitting light to the distal end 8 of the shaft to illuminate the anatomy being viewed through the guidewire.

The optical fiber may be of 10K thickness to provide a resulting guidewire diameter of approximately 0.035 inch. Alternatively, the optical fiber may be a smaller or larger size to yield a guidewire of a smaller or larger diameter. The light fiber bundle 12 may be any fiber element suitable for transmitting light from a fiber optic light source.

The jacket 7 is preferably a preformed tubular structure formed from a polymer such as polyimide. To assemble the guidewire shaft 4, the optical fiber 10 and light fiber bundle 12 are loaded into the jacket 7 and the ends of the fibers and the jacket are secured by adhesive to maintain them in connection. Ideally, individual fibers 14 of the light fiber bundle 12 are separated apart as they approach the distal end 8 of the shaft. Preferably, the individual fibers 14 are spread out to surround the optical fiber 12, which is positioned centrally within the lumen 11 of the shaft. The fiber elements may then be bonded by adhesive to maintain this position. The circumferential pattern of light bundle elements 14 will thus provide a more even field of light to illuminate objects being viewed through the optical fiber 10. However, the light fiber bundle 12 may comprise any number of fibers and may be arranged in any configuration determined to be useful for illuminating a given procedure. However, a design consideration relating to configuration of the fibers is the outside diameter of the resulting guidewire.

The shaft 4 may be formed from various materials to provide alternate flexibilities in the resulting guidewire. For example, more flexible or more rigid polymer materials may be formed into the tubular jacket shaft, which surrounds the fibers. Alternatively, a stainless steel hypotube material may be used to enhance structural rigidity of the guidewire. Another alternative for forming the jacket is to apply a polymer material as a coating over the fiber elements 10 and 12.

Releasably attached to the proximal end 6 of the shaft 4 is an optical handle 20 containing image output means such as a series of lenses or digital converter (not shown) through which is transmitted the image from the optical fiber 10 so that it may be seen through an such as an eyepiece 24 at the proximal end 26 of the optical handle or electronic video equipment operatively connected to the proximal end of th optical handle. The optical handle further comprises a main body 28, which houses the lenses and is segmented into two portions 30 and 32, in threaded engagement to move telescopicly when rotated relative to each other to adjust the relative positioning of the lenses and, thus, adjust the focus of the image transmitted through them. Each portion 30 and 32 may have etched into its outer surface, a plurality of grasping grooves 34.

The optical handle 20 also comprises a releasable securement mechanism 38, which is releasably securable to the main body 28 by a friction fit between a stem 40 of the securement mechanism and inside surface 42 of body portion 32. A friction fit may be aided by the presence of rubber gasket 44 placed over the stem 40 to provide a sealed engagement and help keep contaminates from reaching the lenses of the handle. On the proximal end 46 of the securement mechanism 38 is a releasable securement mechanism such as a threaded collet assembly 48, shown in FIG. 1.

The threaded collet assembly comprises several tapered jaws 50 that are compressed together to reduce diameter through hole 52 when a tapered nut is advanced on threads 54 to compress the jaws 50. Collet nut 58 is threaded onto the collet assembly 48 after the optical fiber 10 has been passed through hole 60 of the nut and through hole 52 of the securement mechanism. During relative rotation of the collet 48 and the nut 58 to threadably engage them, the collet may be grasped at raised thumb wheel 49. Advancement of the nut 58 then serves to compress jaws 50 around the optical fiber 10 to secure it to the optical handle.

The collet nut 58 also serves as a platform for the quick connect light post assembly 62, which joins a cable 64 from a fiber optic light source 66 to the light fiber bundle 12 so that light may be transmitted to the distal end 8 of the guidewire. The light post 62 may be secured by a friction fit onto a receptacle formed on the nut 58 or by a twist and lock keyway engagement configuration. The light post 62 is additionally shown in phantom in FIG. 1 to demonstrate that it is removed from the nut 58 in order to load the light fiber bundle through its hollow interior so that the proximal tip 70 of the light bundle protrudes slightly from the light post when mounted on the nut 58. A slot 74 along the top surface of the nut 58 permits the light fiber bundle to slip into the interior 61 through hole 60 of the nut 58 when the light post 62 is again connected to the nut during loading. A quick connect cap 78 is provided at the end of the fiber optic light source cable 64 so that a quick connection with the light post can be made after the optical handle has been assembled with the shaft 4 of the guidewire. The quick-connect cap 78 may be secured to the light post 62 by friction fit, snap fit provided by optional ridge 63, or by threaded engagement (not shown).

In the fully assembled condition, the jacket 7 of the shaft 4 resides loosely through hole 60 of the nut 58. The proximal tip 80 of the optical fiber 10 should be adjusted to protrude slightly from the stem 40 of the securement mechanism 38 before the nut 58 is tightened to lock it in place. A slight distance of protrusion will place the proximal tip 80 of the optical fiber within close distance from the lenses of the main body 28 without contacting it. It is important that relative movement between the proximal end 80 of the fiber and the lens of the handle be restrained to prevent damage. It is preferred that the securement mechanism grasp at least the fiber 10 rather than the entire shaft 6 by the outer jacket 7, for more secure engagement. Grasping the jacket may permit some relative movement of the fiber, if it too is not also secured. However, the jacket of the shaft may be grasped by the mechanism, if restraint of the fiber can be controlled.

After assembly of the optical handle with the shaft 4 of the guidewire, the guidewire may be used in navigating passageways and organs of the body providing viewing capability from its distal face 13 as transmitted from the optical fiber 10. The range of view of the guidewire arranged in this manner is approximately 5 centimeters. The images transmitted by the optical fiber may be viewed directly at the eyepiece 24 of the optical handle. Alternatively, the optical handle may be coupled to a video camera lens 91 by a threaded eyepiece connector 90. As shown in FIG. 1, the connector 90 has a front flange 92 incorporating a U-shaped bracket 94 that holds the eyepiece. External threads 96 on the back of connector 90 engage internal threads 98 found on the lens 91 of most cameras. As the connector is tightened with the camera lens threads 98, the eyepiece 24 becomes wedged against the camera lens 91, bracket 94. Use of a video camera permits the user to display the image on a video monitor, and otherwise manipulate the image to enhance viewability such as zooming and focusing.

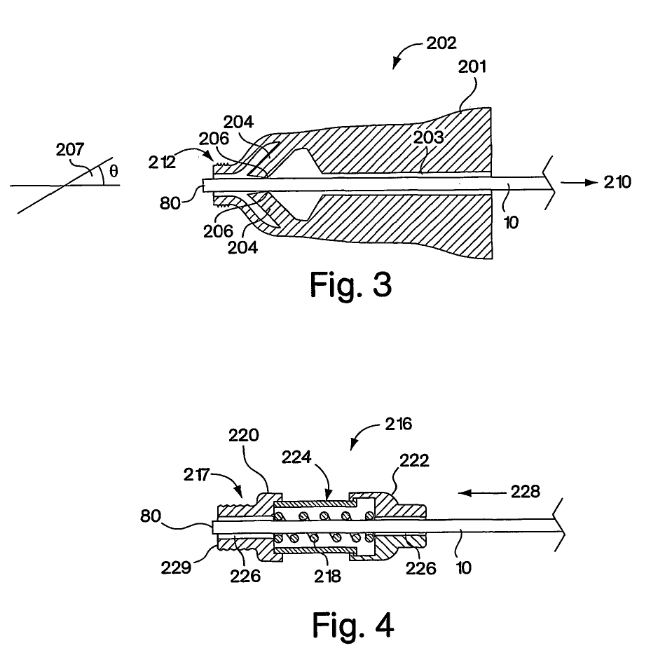

FIGS. 3-9 show alternate designs for the releasabl securement mechanism engaging the optical fiber bundle 10. The alternate configurations may be integrated into the optical handle assembly described above, replacing collet assembly 48. Each securement mechanism embodiment receives the proximal end of the optical fiber 10, and serves as a fixture that secures the fiber from relative longitudinal or lateral movement and is itself releasably retainable in the inside surface 42 of the optical handle.

FIG. 3 shows sectional view of a releasable securement mechanism 202 comprising a body 201 with an internal passage 203 sized to closely receive the optical fiber 10. Along the passage 203 one or more resilient spring fingers 204 are arranged to meet and engage the proximal end of the optical fiber 10 at an acute angle θ 207 and such that locking distal tips 206 of the fingers engage the shaft 10 to lock it in position upon application of a withdrawal force in the distal direction 210 upon the shaft. The fingers 204 bend radially outward when engaged by a proximally advancing fiber to permit loading. The passage 203 provides lateral stability to support the fiber. A stem 212 is provided at the proximal end of the body 201 to engage the inside surface of the optical handle as with the embodiment described above.

FIG. 4 shows a sectional view of another alternate releasable securement mechanism 216. The mechanism comprises a body 217 adapted to receive the proximal end of the fiber 10 through a flexible helical spring 218 configured to increase in diameter upon compression to permit entry of the fiber shaft 10 and to return to a reduced diameter in a relaxed position, which reduces the diameter of the spring such that the spring engages the shaft 10 to lock the shaft in position. The spring is loosely contained in central portion 224 of the body 217, with room to expand when compressed. The spring is compressed between proximal portion 220 of the body and distal portion 222, which is joined to but longitudinally slidable relative to the proximal and central portions 220 and 224 of the body. When compressive force is applied (in the proximal direction 228 against distal portion 222, the spring 218 is compressed and expands to permit entry of the fiber shaft 10 through the body. Guide channels 226 in the proximal and distal portions 220 and 222 are sized only slightly larger that the diameter of the fiber and serve to provide lateral stability to the fiber. When compressive force is released, the spring relaxes as it elongates and its diameter reduces to engage the fiber shaft 10, securing it in position win the body 217. As with the other embodiments, a stem 229 protrudes from the proximal portion 220 of the body 217 to engage the inside surface 42 of the optical handle.

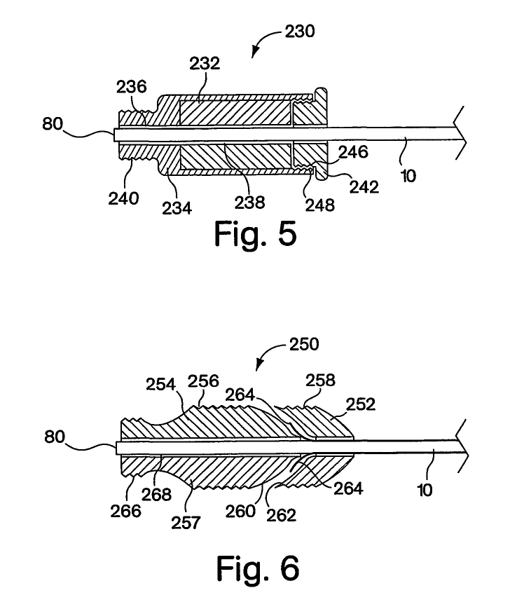

FIG. 5 shows a sectional illustration of another alternate securement mechanism 230 comprising a Toughy-Borst type connector 232. The Toughy-Borst is a cylindrical segment of pliable resilient material, such as silicon, that when is compressed longitudinally, expands laterally, resulting in a reduction of the diameter of its central passage 238. Body 231 of the mechanism contains the Toughy-Borst connector 232 such that central passage 238 is in alignment with body channels 236 to receive fiber shaft 10. Also the body 231 comprises means to apply longitudinal force on the Toughy-Borst connector, such proximal and distal telescopic segments 234 and 242, which may be advanced longitudinally relative to each other by their threaded relationship when they are rotated. Threads 246 and 248 engage to cause relative longitudinal movement to compress or release the Toughy-Borst connector maintained between the proximal and distal portions 234 and 242. As with the other embodiments, a stem 240 protrudes from the proximal portion 234 of the body 231 to engage the inside surface 42 of the optical handle.

FIG. 6 shows another embodiment 250 of the releasable securement mechanism. In this embodiment the shaft further includes a flared segment 264 adjacent its proximal end. The body 254 is configured to grip the flared segment. The flared segment may comprise the proximal end of jacket 7 of shaft 6 rather than an attachment point directly on fiber 10, as with the previous embodiments. The body 254 comprises an inside engagement surface 260 to contact an inside surface of the flange 264 and an outside engagement surface 262 to contact an outside surface of the flange arranged distal to the inside surface 260. The surfaces are moved together to grip the flare 264 therebetween. Surfaces may be portions of two separate sections 257 and 252 threadably engageable by threads 256 and 258 on each. As distal portion 252 is advanced toward proximal portion 257 by rotation, the flare 264 becomes captured between surfaces 260 and 262. Central passage 268 of the body 254 is sized to closely receive the fiber 10 to provide lateral support. As with the other embodiments, a stem 266 protrudes from the proximal portion 257 of the body 254 to engage the inside surface 42 of the optical handle.

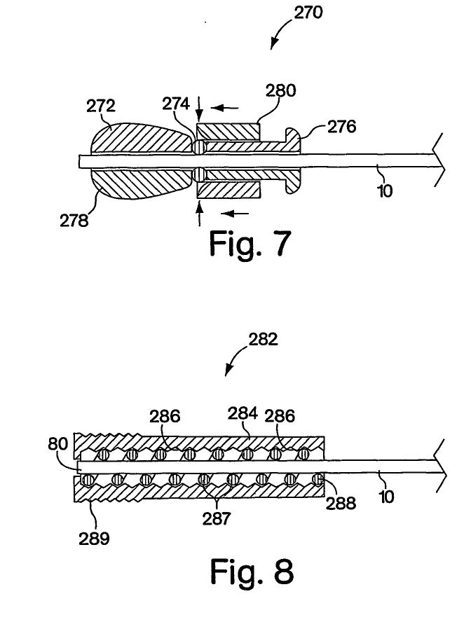

FIG. 7 shows a sectional view of another embodiment 270 of the releasable securement mechanism. The embodiment comprises a body 272 holding a compressible ring 274 about a longitudinal passage 276 through which the fiber 10 passes. The body also receives an outer slidable member 280 having a first position and a longitudinally spaced second position. In the first position, the compressible ring is not engaged by the slidable member 280 and is released to be in a relaxed position to define an inside diameter that is greater than the diameter of the fiber shaft 10. In a second position, the slidable member 280 is moved proximally along the body 272 and engages the compressible ring to apply a compressive force that reduces its inside diameter to engage the shaft.

FIG. 8. shows a sectional view of another alternate embodiment 282 of a releasable securement mechanism. The embodiment comprises a sleeve 284 having internal threads 286 and a helical spring 288 engageable with the internal threads of the sleeve and with the fiber shaft 10. The flexible helical spring may be compressed slightly to increase its diameter to permit loading onto the proximal end of the fiber shaft 10. Next the sleeve 284 is threaded over the coil, its threads 286 engaging the helical arrangement of coils 287 of the spring to become threadably engaged. The diameter of the sleeve is sized to slightly compress the spring when in engagement with it. The slight compression causes the coils 287 to engage and lock the fiber shaft 10. Proximal end 289 of the sleeve may serve as a stem to engage inside surface 42 of the optical handle.

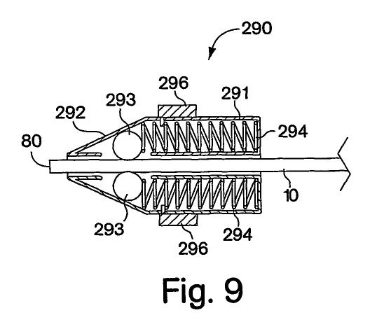

FIG. 9 is a sectional view of another alternate embodiment 290 of the releasable securement mechanism. The mechanism comprises a sleeve 291 having a ramped surface 292 at one end and one or more slidable shoes or balls 293 resiliently biased against the ramped surface to be directed radially inward, into contact with the shaft of the fiber 10. The shoes may be biased by springs 294. The biasing force of the springs may be temporarily removed to permit release of the mechanism from the shaft by sliding catches 296, attached to springs, along surface of the sleeve to compress the springs. Though not shown, the outside surface at the proximal end 297 of the mechanism 292 may be configured as a stem shape to engage the inside surface 42 of the optical handle.

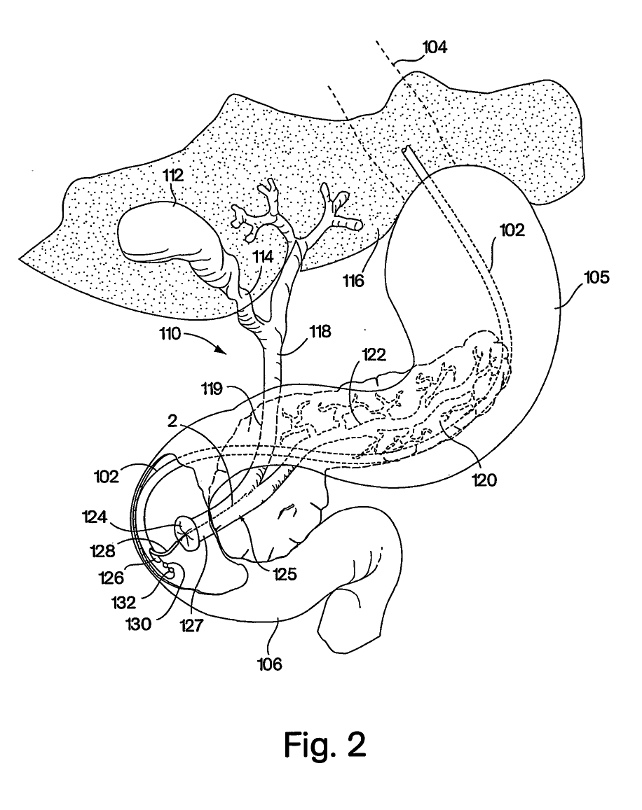

FIG. 2 shows an example of how the guidewire having viewing capability may be used in a biliary procedure. An endoscope 102 having viewing capability and a working channel is first navigated down the esophagus 104 of a patient. The endoscope is advanced through the stomach 105 and into the duodenum 106 at the bottom of the stomach. The biliary tree 110 comprises the cystic duct 114 from the gall bladder 112, the hepatic duct 118 from the liver 116 and the pancreatic duct 122 from the pancreas 120. Each of these ducts joins into the common bile duct 119. The common bile duct 119 intersects with the duodenum a slight distance below the stomach. The papilla (sphincter muscle) 124 controls the size of the opening at the intersection between the bile duct 119 and duodenum.

The papilla must be crossed by a biliary device in order to reach the common bile duct to perform a biliary procedure. The endoscope is navigated so that the side exit port 126 is directly across from the papilla (short endoscope position) or so that the port is slightly below the papilla (long endoscope position) in instances where the duodenum length from the stomach to the biliary tree is relatively short, which can cause the distal end of the endoscope to inadvertently catch the bottom curve of the stomach rather than navigate into the duodenum.

After positioning the endoscope such that the side port 126 of the working channel is adjacent the papilla 124, a biliary catheter 128 is advanced through the working channel the endoscope such that the distal end of the catheter emerges from the side port 126 of the endoscope. Side viewing port 130 and light 132 of the endoscope are arranged to provide viewing of the catheter 128 as it emerges from the endoscope and is moved to enter the papilla 124. Typically, a cannula is first navigated into the papilla. However, the guidewire 2 of the present invention may be preloaded into the cannula. The added viewing capability provided by the present guidewire aides in navigating to and through the papilla. After crossing the papilla, the cannula is advanced into the common bile duct along with the guidewire. The guidewire may be advanced further into the bile duct for examination of abnormalities in the duct or exploration of the pancreas, liver or gall bladder. After the initial cannulation, the guidewire may be left in place in the common bile duct to provide a track over which other devices may be easily introduced, such as a papillatome or stone balloon catheter.

To exchange a catheter over the guidewire, the optical handle 20 must first removed from the proximal end 6 of the shaft. Reference is made to the embodiment of FIG. 1 for illustration purposes. The releasable connector may be disconnected from the main body 28 of the optical handle to facilitate handling and disassembly. The light source cap 78 is disconnected from the light post 62, and the post snapped out of position to remove the light bundle. Next, the collet nut 58 may be rotated to loosen the collet 48 and the securement mechanism may be removed from the optical fiber 10. The proximal end of the shaft is then free to pass a catheter being withdrawn from the wire and can receive a new catheter to be advanced into position. After the catheter exchange, the optical handle may be reattached in the reverse order the disassembly steps. To accomplish exchanges using conventional over-the-wire type catheters, the guidewire may be made in a longer exchange length, such as 250 to 450 cm. Alternatively, rapid exchange catheters may be used with the guidewire to facilitate the exchange.

It should be understood however, that the foregoing description of the invention is intended merely to be illustrative thereof and that other modifications, embodiments and equivalents may be apparent to those who are skilled in the art without departing from its spirit. Having thus described the invention what we desire to claim and secure by Letters Patent is:

1. A guidewire having viewing capability comprising:
a shaft comprising an optical fiber, light transmission fiber bundle, and a jacket surrounding the optical fiber and the light bundle; and
a detachable optical handle having an image output means and
a releasable securement mechanism for selectively attaching the handle to the proximal end of the shaft to permit transmission of image transmitted through the optical fiber to the image output means.

2. A guidewire as defined in claim 1 wherein the releasable securement mechanism further comprises a collet threadably engageable with a nut to provide a releasable clamping force on at least the optical fiber of the shaft.

3. A guidewire as defined in claim 1 wherein the releasable securement mechanism comprises resilient spring fingers arranged to meet an indwelling shaft at an acute angle relative to a longitudinal axis of the shaft and such that locking distal tips of the fingers engage on at least the optical fiber of the shaft to lock it in position upon application of a withdrawal force upon the shaft.

4. A guidewire as defined in claim 1 wherein the releasable securement mechanism comprises a flexible helical spring configured to increase in diameter upon compression to permit entry of the shaft and to return to a reduced diameter in a relaxed position which reduces the diameter of the spring such that the spring engages on at least the optical fiber of the shaft to lock the shaft in position.

5. A guidewire as defined in claim 1 wherein the releasable securement mechanism comprises a Toughy-Borst type connector.

6. A guidewire as defined in claim 1 wherein the shaft further comprises a flared proximal end, the releasable securement mechanism comprises an inside engagement surface and an outside engagement surface movable relative to each other to capture the flange therebetween.

7. A guidewire as defined in claim 1 wherein the releasable securement mechanism comprises a compressible ring engageable with an outer slidable member having a first position and a second position wherein:
in the first position, the compressible ring is released to be in a relaxed position to define an inside diameter that is greater than a diameter of the shaft; and
a second position at which the slidable member engages the compressible ring to apply a compressive force that reduces its inside diameter to engage the on at least the optical fiber of the shaft.

8. A guidewire as defined in claim 1 wherein the releasable securement mechanism comprises a sleeve having internal threads and a helical spring engageable with the internal threads of the sleeve and with at least the optical fiber of the shaft.

9. A guidewire as defined in claim 1 wherein the releasable securement mechanism comprises a sleeve having a ramped surface at one end and one or more slidable shoes resiliently biased against the ramped surface to be directed radially inward, into contact with at least the optical fiber of the shaft the shaft of the guidewire.

10. A guidewire having viewing capability comprising:
an optical fiber;
a light transmission fiber bundle;
a jacket surrounding the optical fiber and light fiber bundle to form an elongate shaft having proximal and distal ends.

11. A guidewire as defined in claim 10 wherein the jacket comprises a polymer sleeve.

12. A guidewire as defined in claim 11 wherein the polymer sleeve comprises polyimide.

13. A guidewire as defined in claim 10 wherein the jacket comprises stainless steel hypotubing.

14. A guidewire as defined in claim 10 wherein the jacket comprises a coating.

15. A guidewire as defined in claim 10 further comprising an optical handle mounted on the proximal end of the shaft.

16. A guidewire as defined in claim 15 wherein the optical handle is releasably attached to the proximal end of the shaft.

17. A guidewire as defined in claim 10 further comprising one or more pressure transducers connected to individual fibers of the optical fiber at the proximal end of the shaft.

18. A method of viewing internal anatomy of a patient comprising:
providing a guidewire having viewing capability;
inserting the guidewire into a patient;
viewing internal anatomy through a viewing portal at the proximal end of the guidewire.

19. A method of viewing internal anatomy as defined in claim 18 further comprising the step of placing the guidewire through a lumen of a catheter.

20. A method of viewing internal anatomy as defined in claim 18 further comprising the step of placing the guidewire through a working channel of an endoscope.

21. A method of viewing internal anatomy as defined in claim 20 further comprising the step of viewing an image transmitted from the guidewire and viewing a second image transmitted from a distal end of the endoscope.

22. A method of viewing the internal anatomy as defined in claim 18 further comprising the step of placing the guidewire through a mother/daughter scope combination.

23. A method of viewing the internal anatomy as defined in claim 22 further comprising the steps of viewing a first image, transmitted through the guidewire, viewing a second image transmitted through the daughter scope, viewing a third image transmitted through the mother endoscope.

24. A method of viewing internal anatomy of a patient comprising:
providing a guidewire having viewing capability;
inserting the guidewire into a passageway of a patient;
navigating the guidewire through the passageway and to the anatomical region under study.

25. A method of viewing internal anatomy as defined in claim 24 further comprising placing the guidewire through lumen of a separate medical instrument.

26. A method of viewing internal anatomy as defined in claim 24 further comprising the step of inserting the guidewire through a working channel of an endoscope.

27. A method of examining a passageway of a patient comprising:
providing a guidewire having viewing capability, proximal and distal ends and a releasable optical handle at its proximal end;
providing a first catheter having at least one lumen;
providing a second catheter having at least one lumen;
advancing the guidewire and first catheter to a passageway of a patient with the guidewire positioned within the lumen of a catheter;
removing the optical handle from the proximal end of the guidewire;
withdrawing the first catheter from the passageway of the patient, over the guidewire, while maintaining the guidewire in position;
removing the first catheter from the guidewire;
placing the proximal end of the guidewire in the lumen of the second catheter;
advancing the second catheter over the guidewire into the lumen.

28. A method of examining a passageway of the patient as defined in claim 27 wherein the guidewire is of a length of approximately 250 to 450 centimeters.

29. A method of examining a passageway as defined in claim 27 wherein the guidewire has an outside diameter of approximately 0.035 inch.

30. A method of examining a passageway of the patient as defined in claim 27 wherein the catheters are rapid-exchange type catheters.

31. A method of obtaining multiple pressure measurements simultaneously at different locations along a passageway of a patient comprising:
providing a guidewire having an optical fiber surrounded by a jacket to form an elongate shaft having proximal and distal ends and multiple pressure sensors joined to the distal end of the shaft and connected to individual fibers of the optical fiber;
joining individual fibers of the optical fiber to pressure transducers at the proximal end of the shaft;
navigating the guidewire to a passageway of a patient;
positioning the guidewire so that the several pressure sensors are positioned at desired locations in the passageway;
monitoring pressure measurements obtained from the guidewire.

32. A method of cannulating the papilla of a patient comprising:
providing a guidewire having viewing capabilities;
providing an endoscope having viewing capabilities;
placing the endoscope through the esophagus and duodenum of a patient to be adjacent to the papilla;
inserting the guidewire through a working channel of the endoscope so that it projects from a distal end of the endoscope;
obtaining visual contact with the papilla through the guidewire;
advancing and directing the guidewire into the papilla using visual guidance.

33. A method of cannulating the papilla defined in claim 32 further comprising:
providing a catheter having at least one lumen;
inserting the catheter through a working channel of an endoscope so that it protrudes from the distal end of an endoscope;
advancing the guidewire through the lumen of the catheter so that it projects from the distal end of the catheter;
obtaining visual contact with the papilla through the guidewire;
directing the guidewire into the papilla under visual guidance obtained through the guidewire and by directing the guidewire with the catheter.

34. A method of positioning a stent in a passageway of a human body comprising:
providing a guidewire having viewing capability;
providing a stent mounted on a stent delivery catheter,
navigating the guidewire to a treatment site within a passageway;
navigating the stent delivery catheter over the guidewire to the treatment site;
visually determining the proper position for the stent;
positioning the stent delivery catheter and stent based on visual observation of the proper position;
delivering the stent to the treatment site in the passageway;
positioning the guidewire to visually verify proper positioning of the stent in the passageway.
